(19) United States
(12) Patent Application Publication
(10) Pub. No.: US 2014/0228960 A1
(43) Pub. Date: Aug. 14, 2014

(54) CERVICAL IMPLANT FOR DOGS

(71) Applicants: University of Bern, Bern (CH); Rita Leibinger GmbH & Co. KG, Neuhausen ob Eck (DE)

(72) Inventors: Franck Forterre, Bern (CH); Rita Leibinger, Muehlheim (DE); Enrico Wirth, Donaueschingen (DE)

(73) Assignees: University of Bern, Bern (CH); Rita Leibinger GmbH & Co. KG, Neuhausen ob Eck (DE)

(21) Appl. No.: 14/176,904

(22) Filed: Feb. 10, 2014

Related U.S. Application Data
(60) Provisional application No. 61/812,740, filed on Apr. 17, 2013.

(30) Foreign Application Priority Data
Feb. 11, 2013 (DE) 10 2013 202 211.5

(51) Int. Cl. A61F 2/44

(52) U.S. Cl. CPC A61F 2/442
USPC 623/17.16

(57) ABSTRACT

A cervical implant for dogs includes a plate section for connecting two adjacent vertebral bodies and an intervertebral section which replaces the vertebral disc between the two vertebral bodies, wherein the plate section has at least one receptacle for each vertebral body for fastening the implant.

### CROSS-REFERENCES TO RELATED APPLICATIONS

This application claims the priority of German Patent Application, Serial No. 10 2013 202 211.5, filed Feb. 11, 2013, pursuant to 35 U.S.C. 119(a)-(d), the disclosure of which is incorporated herein by reference.

This application claims the benefit of prior filed U.S. provisional Application No. 61/812,740, filed Apr. 17, 2013, pursuant to 35 U.S.C. 119(e), the disclosure of which is incorporated herein by reference.

### BACKGROUND OF THE INVENTION

The present invention relates to a cervical implant for dogs.

The following discussion of related art is provided to assist the reader in understanding the advantages of the invention, and is not to be construed as an admission that this related art is prior art to this invention.

For fixing vertebral bodies, in particular cervical vertebral bodies, and for stabilizing the cervical spine, usually screws and cement (for example PMMA) are used. It is also known to use plates, which are fastened on the vertebral bodies with screws. The fastening of these implants occurs by means of an invasive procedure, wherein the vertebral end plates are destroyed. In addition these implants pose the risk that the screws penetrate into the spinal cord canal and cause injury to the spinal cord or the nerves. In order to achieve a stable distraction-fusion oftentimes not only one but two implants are used. This not only increases the risk of injury to the spinal cord but also prolongs the time required for the surgical procedure, and the implants can often only be fastened to the vertebral body with difficulty because the space required therefore is oftentimes not available in the cervical vertebral region.

It would therefore be desirable and advantageous to provide a cervical vertebral implant which is simpler to fasten to the vertebral bodies and which allows a certain distraction of the vertebral bodies.

### SUMMARY OF THE INVENTION

According to one aspect of the present invention, a cervical implant for dogs, including a plate section for connecting two adjacent vertebral bodies, and an intervertebral section, which replaces the vertebral disc between the two vertebral bodies, said plate section having at least one receptacle for a bone screw for each vertebrate body for fastening the implant.

In the implant according to the invention, the two vertebral bodies are thus connected with each other and stiffened and via an intermediate vertebral section it is ensured that the two vertebral end plates do not contact one another. The intermediate vertebral section keeps the vertebral bodies at a distance corresponding to the thickness of the vertebral disc.

According to another advantageous feature of the invention the receptacle can have a bore for receiving and supporting the head of the bone screw and the axis of the bore or the screw is adjustable in relation to the plane of the intervertebral section. This creates the possibility to accurately adjust the pate section to the surfaces of the two vertebral bodies to be interconnected so that the screws can be screwed into the vertebral body in the desired direction. The axis can be manually adjusted with or without tools. In particular the receptacle of the plate section is bent into the desired direction so that the implant can be adjusted to the ventral bone surface.

In order to fix the cage on the bone, holes are drilled with a drilling jig in an ideal, preferably V-shaped, angle for fastening with screws. The compression holes on the implant and the spherical head of the screw generate a compression of the vertebral bodies in order to ensure a better growing together.

Advantageously, the intervertebral section has at least one hollow space allowing reducing its weight and enabling bone tissue to grow through the intervertebral section relatively easily. Also the side surfaces of the intervertebral section can have through passages. In order to ensure the stability of the intervertebral section, support webs are arranged in the hollow space. These extend from the center of the intervertebral section radially outwardly and can also be perforated.

Preferably, the intervertebral section is configured concave and convex or biconvex in cranio-caudal direction, wherein one of the narrow sides is connected with the plate section. This configuration allows ventral insertion and fastening of the implant so that a convex side of the intervertebral section points in ventral direction and the other convex side in dorsal direction.

In the implant an opening with threads is provided for filling the implant with bone material. The bone material flows through the provided hollow spaces and becomes distributed evenly. The opening with thread also serves as connection to a guiding rod, which facilitates the positioning of the implant.

In order to achieve an optimal anchoring of the intervertebral section between the two vertebral bodies, the intervertebral section has at its bone plate facing sides anchoring elements in the form of spikes. These prevent a slipping or a wandering of the implant between the two vertebral bodies.

### BRIEF DESCRIPTION OF THE DRAWING

Other features and advantages of the present invention will be more readily apparent upon reading the following description of currently preferred exemplified embodiments of the invention with reference to the accompanying drawing, in which:

FIG. 1 shows a schematic representation of an implant fastened on two vertebral bodies;

FIG. 2 shows a perspective view of the implant; and

FIG. 3 shows a further perspective view of the implant.

### DETAILED DESCRIPTION OF PREFERRED EMBODIMENTS

Throughout all the Figures, same or corresponding elements are generally indicated by same reference numerals. These depicted embodiments are to be understood as illustrative of the invention and not as limiting in any way. It should also be understood that the drawings are not necessarily to scale and that the embodiments are sometimes illustrated by graphic symbols, phantom lines, diagrammatic representations and fragmentary views. In certain instances, details which are not necessary for an understanding of the present invention or which render other details difficult to perceive may have been omitted.

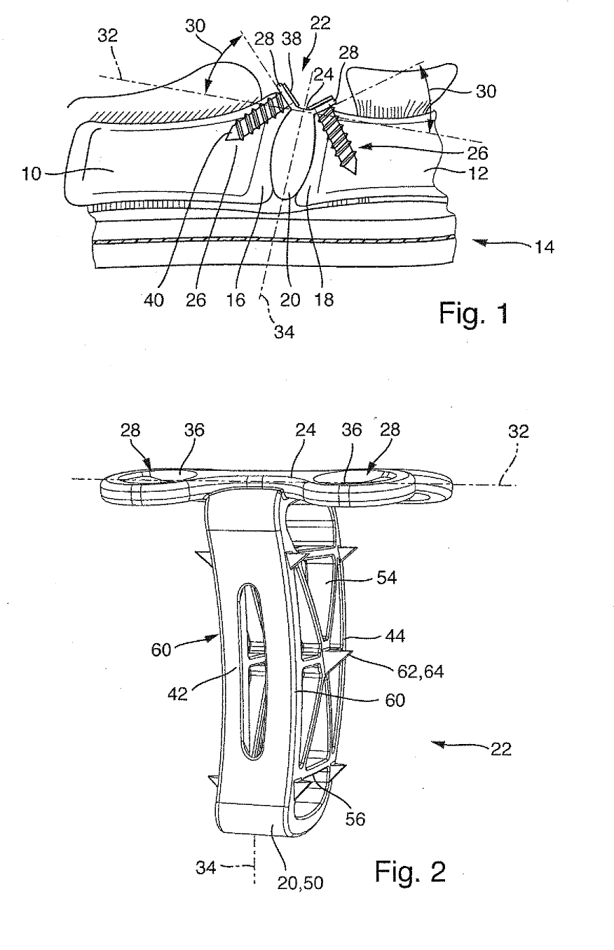

Turning now to the drawing, and in particular to FIG. 1, there is shown a section of a canine vertebral column and the ventral portion of two vertebral bodies 10 and 12 can be seen. Located below these ventral portions is the spinal cord 14. Between the two vertebral plates 16 and 18 of the vertebral bodies 10 and 12 an intervertebral section 20 of a cervical vertebral implant, overall designated with 22, is arranged and supports the two vertebral plates 16 and 18 against each other in the manner of a vertebral disc. For fixing the cervical vertebral implant 22 the latter also has a plate section 24 which is traversed by multiple bone screws 26, wherein the bone screws 26 are screwed into the two vertebral bodies 10 and 12 at the preferred angles. For this, the receptacle 28 is bent by an angle 30 relative to the orthogonal 32 to the plane 34 of the intervertebral section 20.

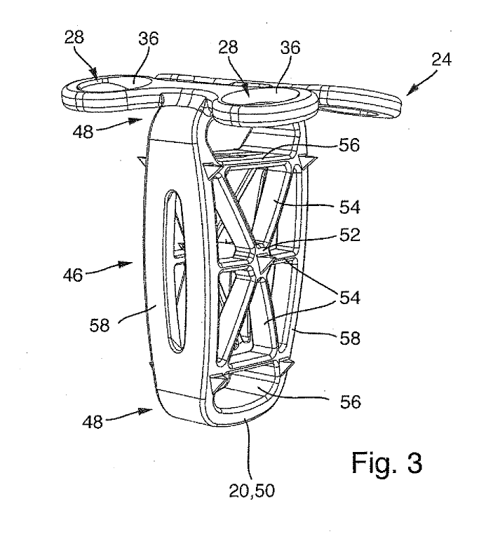

FIG. 2 shows in a perspective view the cervical vertebral implant 22 and the intervertebral section 20 and the plate section 24 can be recognized clearly. The receptacles 28 are still located in the orthogonal 32 and are bent about the desired angle 30 when needed. In addition it can be seen in FIGS. 2 and 3 that the receptacle 38 has a dome-shaped indentation 36 in which the head 38 of the bone screw 26 is located. On its end, which faces the screw shaft 40, the head 38 is configured as a partial sphere so that the indentation 36 forms a pivot bearing for the head 38. In addition, the diameter of the screw shaft 40 is dimensioned so that the head 38 can pivot in the indentation 36 and thereby the screw shaft 40 can pivot relative to the plane of the receptacle 28.

It can also be seen in FIG. 2 that the intervertebral section 20 is configured kidney-shaped and a longitudinal section 42 is configured concave and the second longitudinal section 44 convex. A biconvex configuration is also conceivable. With one of its narrow sides, the intervertebral section 20 is connected one-piece with the plate section 24. It can further be seen, in particular in FIG. 3, that the intervertebral section 20 is configured spherical and has a greater thickness in the center region 46 than in the end regions 48, and that the intervertebral section 20 is configured as hollow body 50.

Located in the hollow body 50 is a central axis 52, from which webs extend radially and two webs 56 are provided which extend parallel to the plate section 24. The webs 54 and 56 are also perforated, as are the concave and convex sidewalls 58. The perforations or hollow configuration of the intervertebral section 20 facilitates growth of bone tissue through the implant 22.

Finally it can also be recognized that the arched or spherical contact surface 60, which comes to abut the two vertebral plates 16 and 18 is provided with anchoring elements 64, which are configured as spikes 62. These spikes 62, which extend pointed, penetrate into the vertebral plates 16 and 18 and prevent a sliding of the intervertebral section 20 and with this sliding of the cervical implant 22.

While the invention has been illustrated and described in connection with currently preferred embodiments shown and described in detail, it is not intended to be limited to the details shown since various modifications and structural changes may be made without departing in any way from the spirit of the present invention. The embodiments were chosen and described in order to best explain the principles of the invention and practical application to thereby enable a person skilled in the art to best utilize the invention and various embodiments with various modifications as are suited to the particular use contemplated.

What is claimed as new and desired to be protected by Letters Patent is set forth in the appended claims and includes equivalents of the elements recited therein:

1. A cervical implant for dogs, comprising:
a plate section for connecting two adjacent vertebral bodies; and
an intervertebral section, which replaces the vertebral disc between the two vertebral bodies, said plate section having at least one receptacle for a bone screw for each vertebrate body for fastening the implant.

2. The cervical implant of claim 1, wherein the receptacle has a bore for receiving and supporting a head of a bone screw and an axis of the bore and/or the bone screw is adjustable relative to the plane of the intervertebral section.

3. The cervical implant of claim 2, wherein the axis is adjustable manually with or without tools.

4. The cervical implant of claim 2, wherein at least regions of the bore are configured dome-shaped and the screw head has a side which faces the shaft, said screw head being configured on said side as a partial sphere.

5. The cervical implant according of claim 2, wherein a diameter of the bore is greater by 5-50% than a diameter the shaft at an end of the shaft proximal to the head.

6. The cervical implant of claim 1, wherein the intervertebral section has at least one hollow space.

7. The cervical implant of claim 6, further comprising support webs arranged in the hollow space.

8. The cervical implant of claim 1, wherein the intervertebral section is configured kidney shaped and a narrow side of the intvertebral section is connected with the plate section.

9. The cervical implant of claim 1, wherein the intervertebral section has a grid structure.

10. The cervical implant of claim 1, wherein sides of the intervertebral section that face the vertebral plates have anchoring elements.

11. The cervical implant of claim 10, wherein the anchoring elements are configured as spikes.

12. The cervical implant of claim 1, wherein the plate section and the intervertebral section are formed integrally or are made in one piece.

13. The cervical implant of claim 1, wherein the plate section and the intervertebral section are formed in two pieces and are fused together prior to fastening to the vertebral bodies.
